(19) United States
(12) Patent Application Publication
(10) Pub. No.: US 2020/0383627 A1
(43) Pub. Date: Dec. 10, 2020

(54) TECHNIQUES FOR TREATMENT OF EPILEPTIC DISORDERS USING ELECTROPHYSIOLOGICAL BIOMARKERS AND RELATED SYSTEMS AND METHODS

(71) Applicant: Children's Medical Center Corporation, Boston, MA (US)

(72) Inventors: Tobias Loddenkemper, Natick, MA (US); Ahmet Tanritanir, Boston, MA (US); Xiaofan Wang, Boston, MA (US)

(73) Assignee: Children's Medical Center Corporation, Boston, MA (US)

(21) Appl. No.: 16/763,953

(22) PCT Filed: Nov. 14, 2018
(86) PCT No.: PCT/US2018/061119

§ 371 Date: May 13, 2020

Related U.S. Application Data
(60) Provisional application No. 62/586,090, filed on Nov. 14, 2017.

(51) Int. Cl. A61B 5/00; A61B 5/048

(52) U.S. Cl. CPC A61B 5/4094; A61B 5/4848; A61B 2503/045; A61B 5/048; A61B 5/7257; A61B 5/7246

(57) ABSTRACT

Techniques for determining treatment of a subject with IS based on EEG-derived biomarkers are provided. According to some aspects, a method of adapting treatment of a subject having infantile spasms (IS) is provided, the method comprising obtaining electroencephalogram (EEG) data of the subject, determining a measure of delta power of the EEG data and/or a measure of spike frequency of the EEG data, and determining subsequent treatment of the infantile spasms of the subject based at least in part on the determined measure of delta power of the EEG data and/or measure of spike frequency of the EEG data.

### CROSS-REFERENCE TO RELATED APPLICATIONS

This application claims the benefit under 35 U.S.C. § 119(e) to U.S. Provisional Application No. 62/586,090, filed Nov. 14, 2017, titled “Electrophysiological and Clinical Biomarkers of ACTH Treatment,” which is hereby incorporated by reference in its entirety.

### BACKGROUND

Infantile spasms (IS), also known as West syndrome, is a rare seizure disorder that occurs in very young children. The seizures include a sudden stiffening, which often cause the arms to fling out as the knees are pulled up and the body bends forward (sometimes called “jackknife seizures”). Less often, the head can be thrown back as the body and legs stiffen in a straight-out position. Movements can also be more subtle and limited to the neck or other body parts. Infants can cry during or after the seizure. Each seizure lasts only a second or two but they usually occur close together in a series. Sometimes the spasms are mistaken for colic, but the cramps of colic do not occur in a series. Children with Infantile spasms often seem to stop developing as expected, or they may lose skills like sitting, rolling over, or babbling.

IS is considered an age-specific epilepsy that typically begin between 3 and 8 months of age. Almost all cases begin by 1 year of age and usually stop by the age of 2 to 4 years. IS is not common, affecting around one baby out of a few thousand. About 2/3 of babies with IS have some known cause for the seizures. A number of conditions may cause changes in the way the brain forms or functions. For example problems with a gene(s) or body metabolism, changes in the brain structure (called a malformation), lack of oxygen to the brain, brain infections or injury before the seizures begin. Others have had no apparent injury and have been developing normally. There is no evidence that family history, the baby's sex, or factors such as immunizations are related to infantile spasms.

### SUMMARY

According to some aspects, a method of adapting treatment of a subject having infantile spasms (IS) is provided, the method comprising obtaining electroencephalogram (EEG) data of the subject, determining a measure of delta power of the EEG data and/or a measure of spike frequency of the EEG data, and determining subsequent treatment of the infantile spasms of the subject based at least in part on the determined measure of delta power of the EEG data and/or measure of spike frequency of the EEG data.

According to some aspects, a non-transitory computer readable medium is provided comprising instructions that, when executed by at least one processor, perform a method of adapting treatment of a subject having infantile spasms (IS), the method comprising accessing electroencephalogram (EEG) data of the subject, determining, using the at least processor, a measure of delta power of the EEG data and/or a measure of spike frequency of the EEG data, and determining, using the at least processor, subsequent treatment of the infantile spasms of the subject based at least in part on the determined measure of delta power of the EEG data and/or measure of spike frequency of the EEG data.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1 is a flowchart of a method of determining treatment of a subject based on EEG-derived biomarkers, according to some embodiments;

FIG. 2 is a schematic showing the basic concept of using Electroencephalography (EEG) to measure the brain activity of a subject;

FIG. 3 is an illustration of the different types of EEG waves, including Beta wave, Alpha wave, Theta Wave, Delta Wave;

FIG. 4A is a flowchart of a method of determining treatment of a subject by comparing EEG-derived biomarkers with previously obtained EEG-derived biomarkers of the same subject, according to some embodiments;

FIGS. 4B and 4C depict illustrative EEG signals obtained, respectively, before and after treatment for infantile spasms, according to some embodiments;

FIG. 5 is a flowchart of a method of determining a likelihood of relapse of a subject based on EEG-derived biomarkers, according to some embodiments;

FIG. 6 is a flowchart of a method of calculating a delta power based on EEG data of a subject, according to some embodiments;

FIG. 7 is a flowchart of a method of calculating a spike frequency based on EEG data of a subject, according to some embodiments;

FIG. 8 is a computing system for gathering and analyzing EEG data of a subject, according to some embodiments; and

FIG. 9 illustrates an example of a computing system environment on which aspects of the invention may be implemented.

### DETAILED DESCRIPTION

Electroencephalography (EEG) is one of the most versatile brain imaging techniques. Electroencephalography non-invasively records electrical activity and brain oscillations using electrodes placed on the scalp. Measuring electrical activity from the brain is useful because it reflects how the many different neurons in the brain network communicate with each other via electrical impulses. To record electrical activity generated by the brain, electrodes may be placed on the scalp surface and the voltages across various electrodes measured. EEG has several benefits compared to other imaging techniques or pure behavioral observations, one of which is its excellent time resolution, that is, EEG can produce hundreds to thousands of snapshots of electrical activity across multiple sensors within a single second. This renders EEG an ideal technology to study the precise time-course of cognitive and emotional processing underlying behavior.

EEG signals obtained from a child with IS typically exhibit very chaotic and disorganized brain electrical activity with no recognizable pattern, termed a “hypsarrhythmia pattern,” whereas comparatively normal brain electrical activity generally exhibits clearly visible patterns. The presence of hypsarrhythmia is often used as a diagnostic criteria for IS, and is characterized by an abnormal interictal pattern consisting of high amplitude and irregular waves and spikes in a background of chaotic and disorganized activity within the EEG signals.

Irrespective of how IS is diagnosed, a number of medications have been employed to treat IS, including Adrenocorticotropic Hormone (ACTH) and Vigabatrin. These medications can be useful for short-term treatment of IS, but many patients with IS relapse. In addition, there is much uncertainty as to which patients will be responsive to the medication. As a result of these issues, effective management of IS can present a challenge.

The inventors have recognized and appreciated techniques for evaluating a subject's response to treatment of IS by evaluating biomarkers present in EEG signals obtained from the subject. The inventors have recognized that values of these biomarkers are highly correlated with a subject's response to treatment of IS and accordingly, when evaluated, can provide feedback on whether past treatment is efficacious and/or be a predictor for relapse. As a result, evaluation of these biomarkers can provide a useful indication of how a subject is responding to treatment of IS and aid in management of the condition.

According to some embodiments, evaluating a subject's response to treatment of IS may be based upon a measurement of delta power of EEG signals obtained from the subject. As will be discussed further below, EEG signals measured from a subject can be broken down into different frequency bands, each of which may be representative of different aspects of the subject's brain function. The delta oscillations are typically defined as the component of the EEG signals with a frequency between 0.5 Hz and 4 Hz, and the inventors have recognized that a measurement of the spectral power of the EEG signal present within the delta frequency band (referred to herein simply as the “delta power”) can be indicative of the subject's response to treatment of IS.

According to some embodiments, evaluating a subject's response to treatment of IS may be based upon a measurement of the frequency of EEG spikes within EEG signals obtained from the subject. As will be discussed further below, EEG signals may contain “spikes,” which are significant deviations from a baseline measurement, typically lasting a short amount of time (e.g., around 100 ms). The inventors have recognized that a measurement of the frequency of such spikes within EEG signals can be indicative of the subject's response to treatment of IS.

Following below are more detailed descriptions of various concepts related to, and embodiments of, techniques for determining treatment of a subject based on EEG-derived biomarkers. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

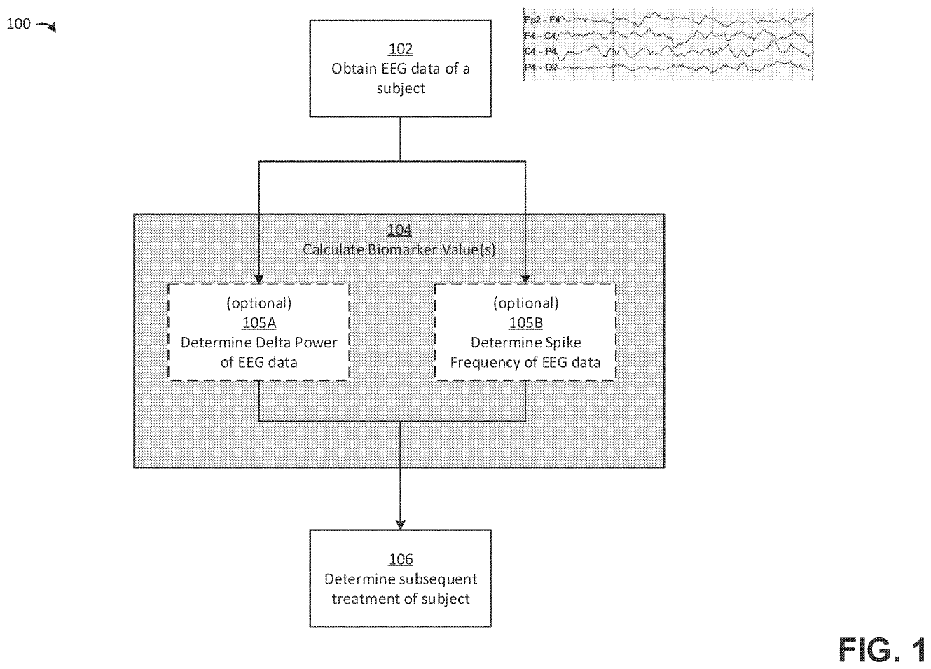

FIG. 1 is a flowchart of a method of determining treatment of a subject based on EEG-derived biomarkers, according to some embodiments. Method 100 calculates one or more biomarker values that are indicative of a degree to which a subject is responding to treatment of IS based on EEG data obtained from the subject. The one or more biomarker values can provide insight into an appropriate subsequent treatment of the subject; for instance, if the values indicate that the subject is responding to the treatment, subsequent treatment may simply maintain prior treatment, or may comprise tapering off or even ceasing treatment. Alternatively, if the values indicate the subject is not responding to the treatment to a desired degree, subsequent treatment may differ from prior treatment by, for instance, supplementing the treatment with a different medication, changing medications, and/or adjusting medication dosage(s).

Method 100 may be performed by any suitable computing device or devices, examples of which are discussed below. In some embodiments, method 100 may be performed by one or more processors of an EEG system. In some embodiments, method 100 may be performed by one or more processors coupled to an EEG system. In some embodiments, method 100 may be performed by one or more computing devices configured to access previously generated EEG data.

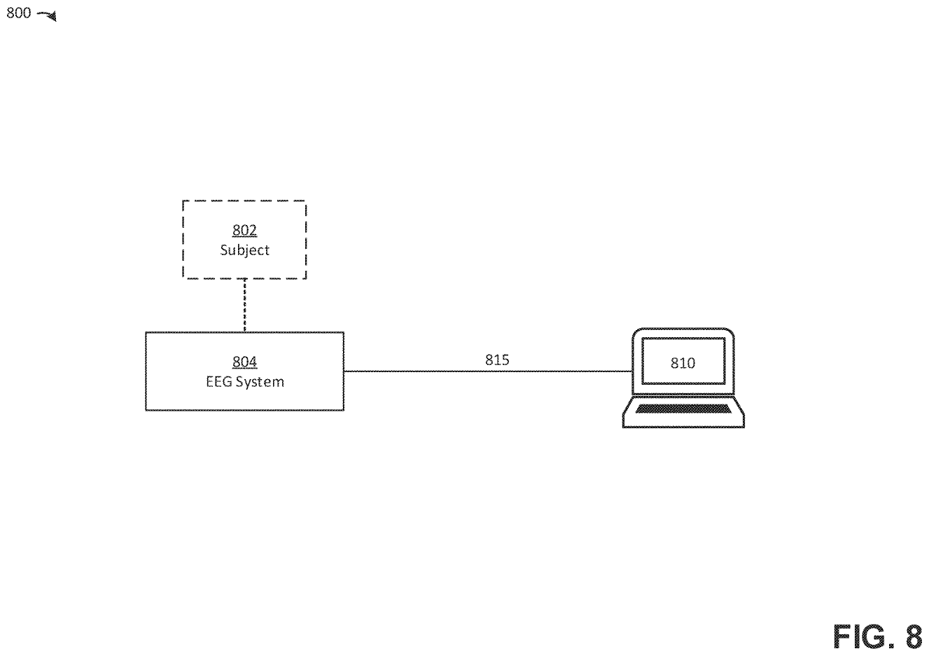

In act 102 of method 100, EEG data of a subject is obtained. The EEG data may, in some embodiments, be obtained by a computing device reading the EEG data from an EEG device in real-time. An example of such a configuration is discussed in relation to FIG. 8 below. In some embodiments, the EEG data obtained in act 102 may have been previously produced by an EEG device and stored on one or more computer readable media and accessed by a computing device performing method 100.

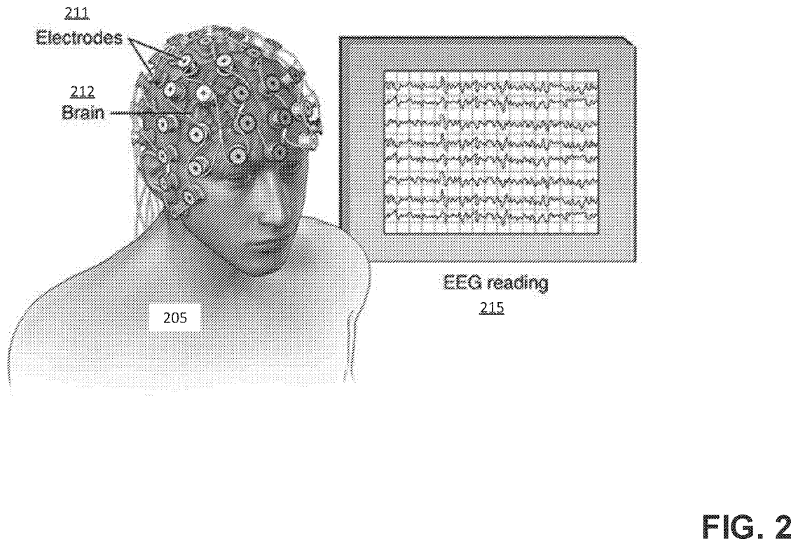

For purposes of illustration, FIG. 2 depicts elements of an EEG system, according to some embodiments. As discussed above, EEG signals are produced by a collection of electrodes, typically comprising somewhere between 10 and 500 electrodes depending on the scope of the analysis. The electrodes 211 are arranged on the scalp and are configured to measure electrical signals produced by the brain 212 of a subject 205. These electrical signals are measured by a suitable device coupled to the electrodes 211 and may be stored and/or analyzed. Typically the electrodes are each connected to an input of an associated amplifier, with a common reference electrode being attached to the other input of each of the amplifiers. The voltages produced at each electrode relative to the common electrode is therefore amplified to a magnitude suitable for analysis. The amplified signals are typically provided to an analog to digital converter for storage and/or analysis of digital data. The resulting amplified, digitized signals, referred to herein as EEG readings or EEG signals, may be recorded and/or displayed as a time series of voltage values 215.

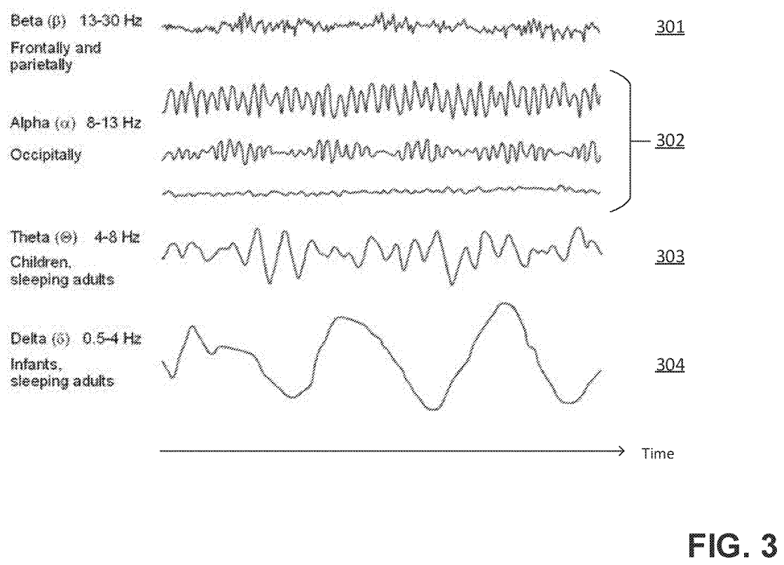

The billions of neurons in the human brain have highly complex firing patterns, mixing in a rather complicated fashion. It is often helpful to view EEG signals as being a mixture of signals with different underlying base frequencies, which are considered to reflect certain cognitive, affective or attentional states. As shown in FIG. 3, which depicts voltage amplitudes produced by an EEG as a time series, EEG signals can be broken up into signals within different frequency bands. These bands are termed the delta band (approximately 0.5 Hz-4 Hz), theta band (approximately 4 Hz-8 Hz), alpha band (approximately 8 Hz-13 Hz), beta band (approximately 13 Hz-30 Hz) and gamma band (greater than around 30 Hz). Examples of EEG signals present within such frequency bands are illustrated in FIG. 3.

EEG signals within the delta band are the slowest and the highest amplitude oscillations, and are generally only present during deep non-REM sleep (stage 3), also known as slow-wave sleep (SWS), and in infants and young children. The amplitude of EEG signals in the delta band (referred to herein as “delta waves”) are stronger in the right brain hemisphere, and the sources of delta waves are typically localized in the thalamus. Since sleep is associated with memory consolidation, delta waves play a core role in the formation and internal arrangement of biographic memory as well as acquired skills and learned information. FIG. 3 depicts an example 304 of a delta wave.

Brain oscillations produced within the approximately 4 Hz-8 Hz frequency band are referred to as theta waves. Studies consistently report frontal theta activity to correlate with the difficulty of mental operations, for example during focused attention and information uptake, processing and learning or during memory recall. Theta frequencies become more prominent with increasing task difficulty. This is why theta is generally associated with brain processes underlying mental workload or working memory. Theta can be recorded from all over the cortex, indicating that it is generated by a wide-ranging network involving medial prefrontal areas, central, parietal and medial temporal cortices. FIG. 3 depicts an example 303 of a theta wave.

Brain oscillations produced within the approximately 8 Hz-13 Hz frequency band are referred to as alpha waves. Alpha waves are generated in posterior cortical sites, including occipital, parietal and posterior temporal brain regions. Alpha waves have several functional correlates reflecting sensory, motor and memory functions. You can see increased levels of alpha band power during mental and physical relaxation with eyes closed. By contrast, alpha power is reduced, or suppressed, during mental or bodily activity with eyes open. FIG. 3 depicts alpha waves 302 in a range of these different states. Alpha suppression constitutes a valid signature of states of mental activity and engagement, for example during focused attention towards any type of stimulus. Alpha suppression indicates that the brain is preparing to pick up information from various senses, coordinating attentional resources and focusing on what really matters in that particular moment.

Brain oscillations produced within the approximately 13 Hz-30 Hz frequency band are referred to as beta waves. Beta waves are generated both in posterior and frontal regions. Active, busy or anxious thinking and active concentration are generally known to correlate with higher beta power. Over central cortex (along the motor strip), beta power becomes stronger as we plan or execute movements, particularly when reaching or grasping requires fine finger movements and focused attention. FIG. 3 depicts an example 301 of a beta wave.

Brain oscillations produced above 30 Hz range are referred to as gamma waves. It is still unclear where exactly in the brain gamma waves are generated and what these oscillations reflect.

Returning to FIG. 1, EEG data obtained in act 102 may comprise EEG signals produced by a suitable EEG device such as that shown in FIG. 2 producing signals from electrodes placed upon a subject. In some cases, the EEG data obtained in act 102 may represent data captured from the subject during a single session (e.g., a continuous period of time during which EEG data is captured from the subject).

In some embodiments, the EEG data obtained in act 102 may include a complete set of time series amplitude data produced by all of the electrodes in an EEG device, whereas in other cases the EEG data may only include such data produced by a portion of the electrodes in an EEG device. Moreover, in some embodiments the EEG data obtained in act 102 may comprise amplitude data produced by an EEG device without particular frequencies having been filtered out of the oscillations. In other cases, the EEG data may comprise amplitude data produced by an EEG device that has been passed through one or more frequency filters, for example to produce EEG signals within one or more frequency bands. Such filtering need not result in a single set of data; for instance, EEG data may comprise EEG signals within the delta band produced by a subject in addition to EEG signals within the alpha band produced by the subject during the same time period as the delta band signals.

In act 104 of method 100, one or more biomarker values are calculated based on the EEG data obtained in act 102. In some cases, a suitable biomarker value may be a single value obtained for a given subject for a given session of the subject producing EEG data. A single value may be useful to provide a single measure of the extent to which the subject is responding to treatment of IS. Multiple single-value biomarkers may each be calculated for the EEG data obtained in act 102 that may each provide at least partially independent indications of the extent to which the subject is responding to treatment of IS. For example, two biomarker values that each represent a different measure of the extent to which the subject is responding to treatment of IS may be calculated in act 104.

In the example of FIG. 1, two illustrative steps 105A and 105B in which a biomarker value is calculated are shown. Either or both of these acts may be performed, and/or different calculations may be performed to produce different values.

In optional act 105A, a delta power is calculated based on the EEG data obtained in act 102. As discussed above, the delta power is a measurement of the spectral power of the EEG signal present within the delta frequency band and can be indicative of the subject's response to treatment of IS. The delta power value can be calculated in various ways, including by generating a power spectral density of EEG data via a Fast Fourier Transform (FFT) analysis. This produces information on the amount of power present in various frequency components. By totaling the power present in the frequencies in the delta band (e.g., 0.5 Hz to 4 Hz), a measure of total delta power may be produced. In some embodiments, such an analysis may be performed by generating power spectral density (PSD) data from EEG data that includes frequencies outside of the delta band; in other embodiments the EEG data obtained in act 102 may have been previously filtered to remove frequencies outside of the delta band, such that the PSD data includes power for only the delta band frequencies.

In optional act 105B, a spike frequency is calculated based on the EEG data obtained in act 102. As discussed above, the spike frequency is a measurement of the frequency of significant deviations from a baseline measurement, which typically lasting a short amount of time. The spike frequency may be calculated in various ways, although primarily by detecting the feature of a spike in some way and totaling up the number of spikes in a given amount of time to determine the frequency. Spikes may be identified in some embodiments via thresholding—that is, by identifying when the amplitude of a brain oscillation passes some threshold. Such a threshold may be specified in some cases as a multiple of (e.g., between 2 and 4 times that of) a baseline amplitude (e.g., a mean amplitude during periods in which spikes do not occur).

According to some embodiments, some or all channels of EEG signals produced by an EEG device may be analyzed to identify times at which spikes occur. It will be appreciated that a spike may occur in any number of channels at a given time, and in some cases may appear in some but not all of the channels. In this case, a single occurrence of a spike may be counted for purposes of identifying the spike frequency. In some embodiments, a band pass filter may be applied to EEG signals prior to analyzing the signals to identify spikes. For example, frequencies present within the EEG signals below 0.1 Hz or above 70 Hz may be filtered out and the resulting filtered signals analyzed to identify spikes.

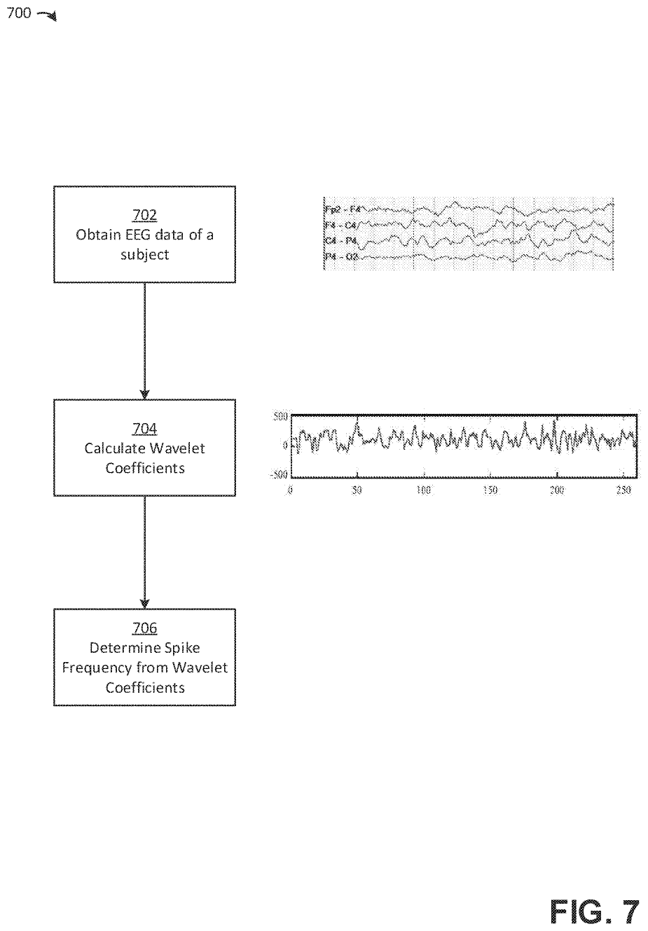

In some embodiments, a spike frequency may be determined through a wavelet analysis in which wavelets are correlated with one or more signals. A wavelet analysis may represent a particularly desirable approach to identifying spikes due to the natural ability of wavelets to identify rapid changes in a signal's amplitude. A more detailed discussion of such an approach is provided below in relation to FIG. 7. In some embodiments, machine learning approaches such as a neural network trained to recognize spikes from an EEG signal or signals may be executed to identify spikes.

It will be appreciated that a spike count may also represent a suitable biomarker value so long as the spike count is produced for a standardized amount of time within the EEG data. For instance, when EEG data is captured over a period of ten minutes, the total spike count during this period may be compared across subjects to determine an extent to which the subject is responding to treatment of IS. As a result, the above-discussed techniques for identification of spikes in the context of spike frequency may also be applicable to a calculation of spike count.

Irrespective of which biomarker value(s) are calculated in act 104, and the particular technique(s) by which they are calculated, in act 106 of method 100 a subsequent treatment for the subject is determined. Since the calculated biomarker value(s) are indicative of an extent to which the subject is responding to treatment of IS, these values may be relied upon to decide what kind of subsequent treatment is appropriate. Subsequent treatment may include maintaining a treatment already being applied to the subject; ceasing treatment of the subject; beginning treatment of the subject (if the subject is currently not under treatment and the biomarker value(s) indicate a likelihood of relapse); or adjusting the treatment by incorporating an additional medication, changing a medication, and/or adjusting medication dosage(s).

According to some embodiments, an identification of a subsequent treatment may be made by a medical professional based on the subject's medical history in addition to the particular value of the biomarker(s) calculated in act 104. As one non-limiting example, a medical professional may determine, when a first subject is not responding to treatment despite having been treated with various different dosages of a particular medication, to change treatment to utilize a different medication, either in addition to, or instead of, the previously applied medication. Alternatively, the medical profession may determine, for a second subject and for identical biomarker value(s) of the first subject, to increase a dosage of medication because the second subject's history is that only a single, comparatively lower dose of the medication has previously been used in treatment.

According to some embodiments, an identification of a subsequent treatment may be made automatically by the computing device or devices performing method 100. In some cases, the biomarker value(s) may indicate the subsequent treatment based on a database of subject outcomes and associated biomarker values. In some cases, a database may also include additional subject information, such as age, weight, prior biomarker values obtained from the subject, baseline biomarker values for the general population or for a similar population to the subject, prior medical history, etc., any one or more of which may be accessed in addition to the biomarker value(s) calculated in act 104 and used along with the biomarker value(s) to determine an appropriate treatment for the subject.

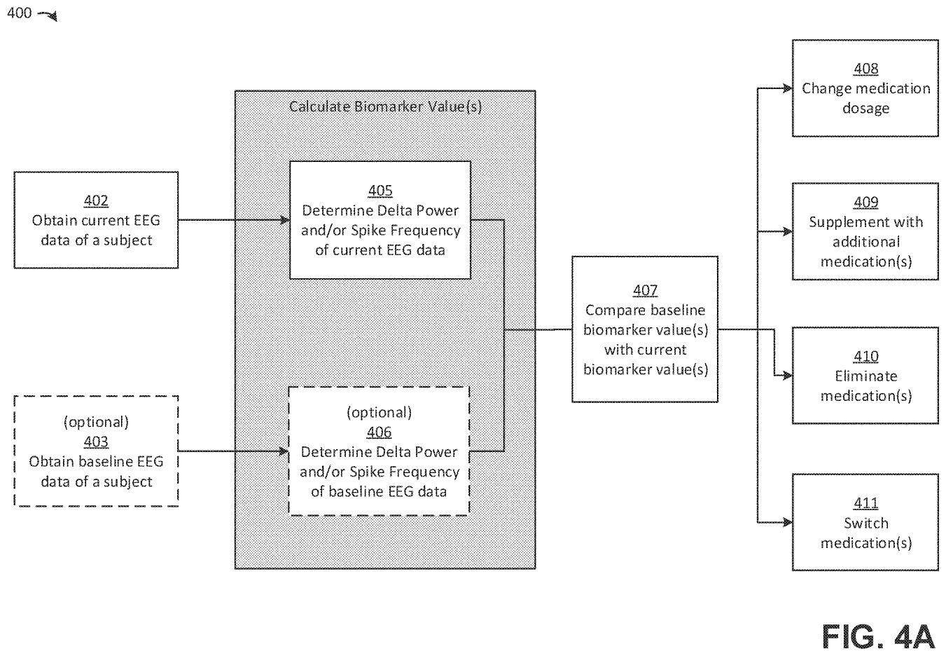

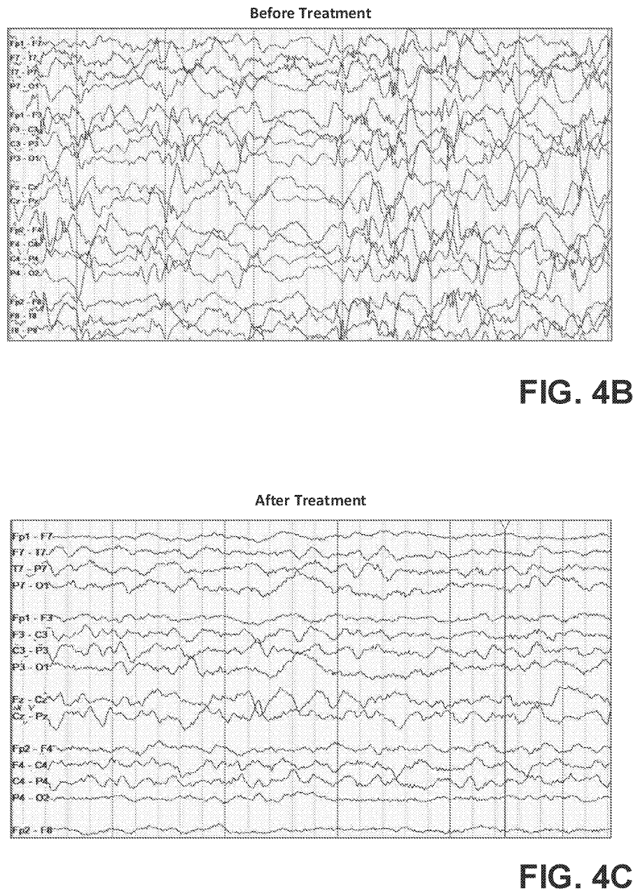

FIG. 4A is a flowchart of a method of determining treatment of a subject by comparing EEG-derived biomarkers with previously obtained EEG-derived biomarkers of the same subject, according to some embodiments. Method 400 illustrates an example of method 100 in which subsequent treatment of IS of a subject is identified by comparing previously-calculated values of biomarkers for the subject (“baseline” values) with newly-calculated values of the same biomarkers for the subject. Such a comparison may aid in determining whether the subject is responding to the treatment by indicating a change in electrophysiological activity over time. As with method 100, method 400 may be performed by a suitable computing device or devices.

In act 402, EEG data of a subject is obtained in any manner described above in relation to act 102 of FIG. 1. The EEG data so obtained is analyzed in act 405 to calculate a delta power and/or a spike frequency of the EEG data as described above in relation to acts 105A and 105B of method 100. The delta power and/or spike frequency may be compared with respective baseline values of the delta power and/or spike frequency in act 407 (i.e., a current delta power may be compared with a baseline delta power, or a current spike frequency may be compared with a baseline spike frequency, or a current delta power may be compared with a baseline delta power and a current spike frequency may be compared with a baseline spike frequency).

Optionally, a baseline EEG of the subject may be obtained in act 403 and the baseline EEG data analyzed in act 406 to calculate a delta power and/or a spike frequency of the baseline EEG data. Acts 403 and 406 are optional in method 400 because act 407 may access previously calculated and stored values of the delta power and/or spike frequency that were calculated for the subject at an earlier time. Thus, acts identical to acts 403 and 406 may have been previously performed in addition to a step in which the calculated value(s) are stored prior to the performance of method 400.

Irrespective of whether acts 403 and 406 are performed in method 400, selection of subsequent treatment of the subject is determined based on a result of comparing the baseline value(s) with the current value(s). For instance, one such result of comparing the baseline value(s) with the current value(s) may be to determine a difference in such respective pairs of values. It has been found by the inventors, for instance, that delta power of a subject with IS generally decreases after treatment, but the degree to which the value decreases provides an indication of how much the subject is responding to the treatment. Illustrative subsequent acts 408, 409, 410 and 411 are depicted in the example of FIG. 4A, and any one or more of acts 408, 409, 410 and/or 411 may be performed any number of times as a result of the comparison made in act 407. Which acts are selected and how they are performed may be, as discussed above, determined according to a database of subject outcomes and associated biomarker values.

In act 408, the dosage of one or more medications that have been previously part of the patient's treatment are changed, which may include lowering the dosage of one or more medications and/or increasing the dosage of one or more medications. In act 409, one or more additional medications not previously been part of the patient's treatment plan (or, at least, not currently part of the patient's treatment plan) is/are selected for inclusion in subsequent treatment. In act 410, one or more additional medications currently part of the patient's treatment plan is/are selected to be eliminated from subsequent treatment. In act 411, one or more medications currently part of the patient's treatment plan are exchanged for one or more other medications for subsequent treatment.

For purposes of illustration, FIGS. 4B and 4C depict two instances of the same set of EEG signals produced from the same subject before treatment for IS (FIG. 4B) and after treatment for IS (FIG. 4C).

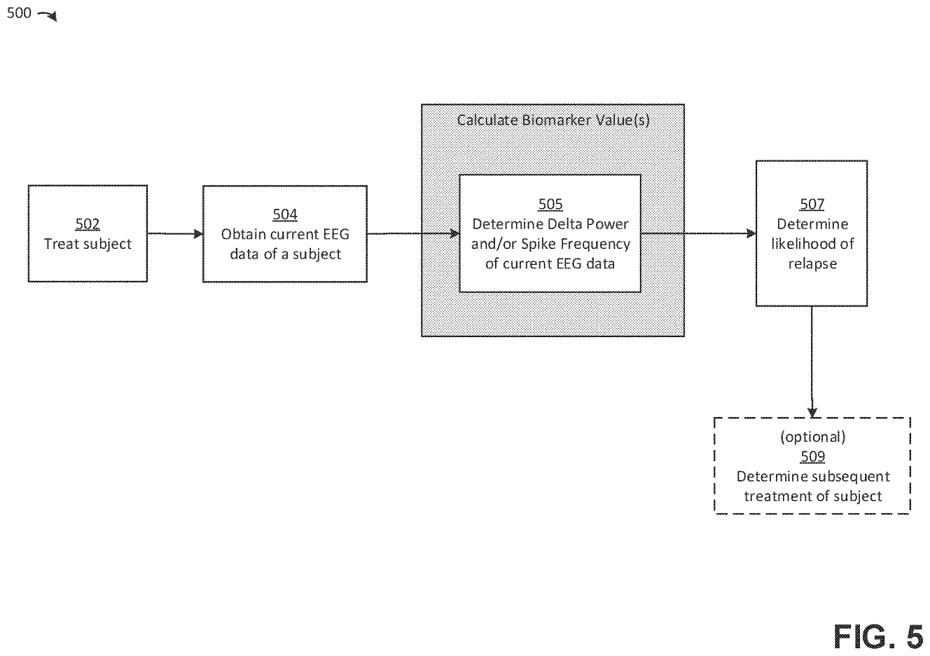

FIG. 5 is a flowchart of a method of determining a likelihood of relapse of a subject based on EEG-derived biomarkers, according to some embodiments. As discussed above, biomarker values calculated according to the techniques herein may be indicative of a likelihood of relapse of IS in the subject. Method 500 depicted in FIG. 5 is an example of a process in which biomarker values are evaluated in this manner. As with method 100, method 500 may be performed by a suitable computing device or devices.

In act 502, a subject is treated for IS via any suitable means. In some embodiments, act 502 may include one or more aspects of methods 100 and/or 400 shown in FIGS. 1 and 4 respectively. That is, treatment of the subject may be determined at least in part based on values of biomarkers calculated as discussed above.

In act 504, EEG data of a subject is obtained in any manner described above in relation to act 102 of FIG. 1. The EEG data so obtained is analyzed in act 505 to calculate a delta power and/or a spike frequency of the EEG data as described above in relation to acts 105A and 105B of method 100. In act 507, a likelihood of relapse may be determined based on the delta power and/or spike frequency determined in act 505. The likelihood of relapse may be, as discussed above, determined according to a database of subject outcomes and associated biomarker values. For instance, it has been found by the inventors that the likelihood of relapse increases with the value of the delta power increases. The likelihood of relapse indicated by the biomarker value(s) determined in act 505 may optionally provide for a determination of subsequent treatment of the subject in act 509. For instance, when a subject is not currently under treatment for IS, but a likelihood of relapse is determined to be comparatively high in act 507, subsequent treatment may include resuming any suitable treatment for IS as a preventative measure.

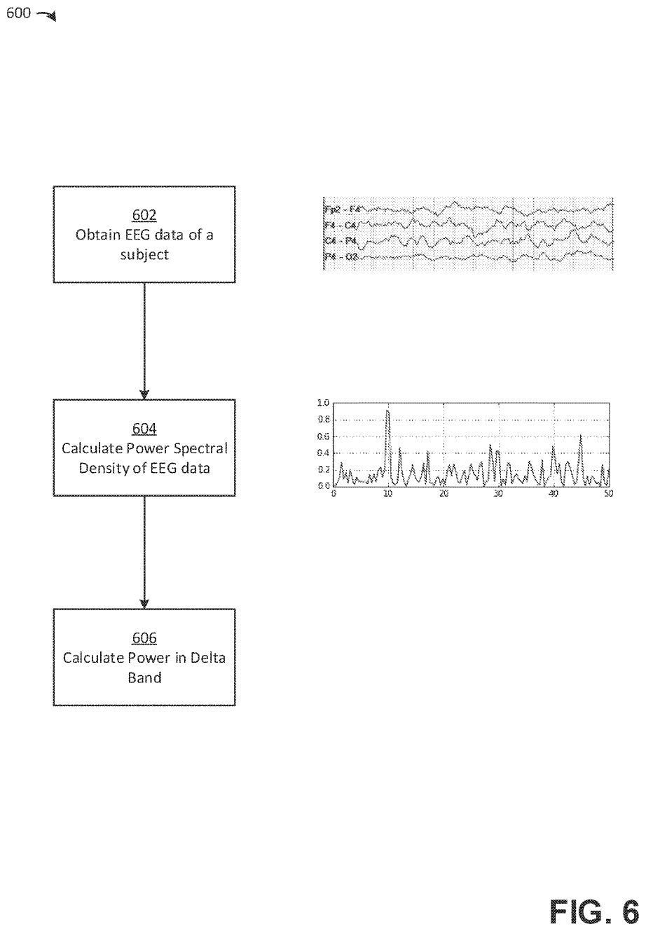

FIG. 6 is a flowchart of a method of calculating a delta power based on EEG data of a subject, according to some embodiments. Method 600 provides one example of calculating a delta power value based on EEG data of a subject and may represent, for instance, acts 102 and 105A in FIG. 1. Method 600 may be performed by any suitable computing device or devices.

In act 602, EEG data of a subject is obtained in any manner described above in relation to act 102 of FIG. 1. In act 604, a power spectral density (PSD) of at least part of the EEG data is calculated. The PSD, as discussed above, provides information on the amount of power present in various frequency components of the EEG signal(s). As one non-limiting example, a Fast Fourier Transform (FFT) of one or more EEG signals may be performed to determine the PSD of the EEG signals. Suitable parameters of the FFT operation may include a time constant of 0.16 seconds, a sampling rate of 64 Hz, and/or an FFT window duration of 2 seconds. In some embodiments, a high-pass, low-pass and/or band-pass filter may be applied to EEG signals prior to generation of a PSD. For instance, frequencies above 35 Hz may be filtered from the EEG signals and a PSD generated based on the resulting filtered EEG signals. It will be appreciated that, because an FFT operates on a window of time of the EEG signal(s), a number of PSDs may be calculated for a number of (overlapping or non-overlapping) windows of time of the EEG signal(s). As a result, act 604 may be performed numerous times.

In act 606, a delta power is calculated based on a PSD calculated in act 604. In cases where a number of PSDs are calculated in act 604, a number of delta powers may be calculated in act 606 by calculating a delta power for each PSD. In some cases, the delta power may vary over time within the EEG signals obtained from a subject in a single session. In these cases, it may be preferable to obtain a single average value of the delta power from the EEG signals by, for instance, calculating the mean of the calculated delta power. Delta power may be calculated by determining the total spectral power present within the calculated PSD data between the frequency bounds of the delta band (e.g., 0.5 Hz to 4 Hz). In some cases, the delta power may be calculated by determining a total area under the PSD curve within the delta band's frequency range.

FIG. 7 is a flowchart of a method of calculating a spike frequency based on EEG data of a subject, according to some embodiments. Method 700 provides one example of calculating a spike frequency value based on EEG data of a subject and may represent, for instance, acts 102 and 105B in FIG. 1. Method 700 may be performed by any suitable computing device or devices.

In act 702, EEG data of a subject is obtained in any manner described above in relation to act 102 of FIG. 1. In act 704, a plurality of wavelet coefficients may be calculated based on the EEG data obtained in act 702. As discussed above, wavelet analysis may represent a particularly desirable approach to identifying spikes due to the natural ability of wavelets to identify rapid changes in a signal's amplitude. Act 704 may comprise any suitable approach to determining wavelet coefficients, including a continuous wavelet transform (CWT), a discrete wavelet transform (DWT) and/or a stationary wavelet transform (SWT). In some embodiments, an SWT analysis may be preferable because the results are invariant with respect to time shifts, although a greater redundancy in data may be produced compared with a DWT analysis, for example. Any suitable wavelet family may be utilized during act 704, including haar, daubechies, biorthogonal, coiflets and/or symlets (e.g., symlet4).

In act 706, spikes are identified from the wavelet coefficients determined in act 704. Act 706 may comprise identifying a number of instances in which the wavelet coefficients have values above or below a suitable threshold that represents an incidence of a spike, to produce a spike count. A spike frequency may be determined by dividing the spike count by the total amount of time represented by the EEG data.

FIG. 8 is a computing system for gathering and analyzing EEG data of a subject, according to some embodiments. System 800 illustrates a system suitable for obtaining EEG signals from a subject and analyzing the signals in real time, and includes an EEG system 804 having a number of electrodes suitable for attachment to a subject 802, and a computing device 810. The computing device 810 is coupled to the EEG system 804 via link 815, which may comprise any suitable wired and/or wireless communications connection. In some embodiments, a single housing holds the computing device 810 and EEG system 804 such that the link 815 is an internal link connecting two modules within the housing of system 800.

According to some embodiments, computing device 810 may execute software that calculates biomarker values from EEG signals produced by the EEG system 804. In some embodiments, the computing device 810 may automatically determine a subsequent treatment for subject 802 based on one or more determined biomarker values. For instance, the computing device 810 may access one or more databases to perform a lookup based on the biomarker value(s), and in some cases further based on data associated with the subject. Such databases may be stored by computing device 810 or stored elsewhere and accessed by computing device 810.

Various parameters and other data associated with analysis of EEG signals, such as algorithms to calculate biomarker values, to perform transformations on EEG signals (e.g., an FFT operation) may be stored by system 800 and accessed when analyzing the EEG signals and calculating values of one or more biomarkers.

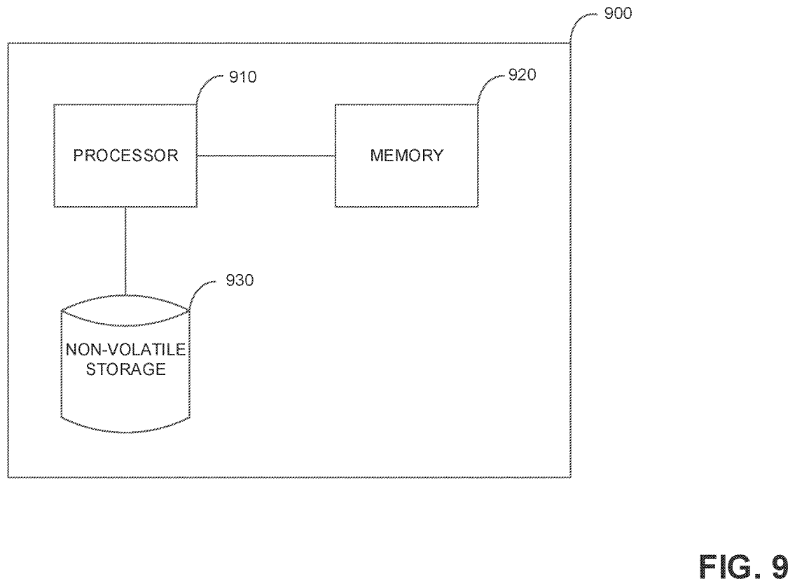

An illustrative implementation of a computing device 900 that may be used to perform any of the techniques described herein is shown in FIG. 9. The computing device 900 may include one or more processors 910 and one or more non-transitory computer-readable storage media (e.g., memory 920 and one or more non-volatile storage media 930). The processor 910 may control writing data to and reading data from the memory 920 and the non-volatile storage device 930 in any suitable manner, as the aspects of the invention described herein are not limited in this respect. To perform functionality and/or techniques described herein, the processor 910 may execute one or more instructions stored in one or more computer-readable storage media (e.g., the memory 920, storage media, etc.), which may serve as non-transitory computer-readable storage media storing instructions for execution by the processor 910.

In connection with techniques described herein, code used to, for example, calculate biomarker values, perform FFTs of EEG signals, perform wavelet transforms, calculate spike frequency, etc. may be stored on one or more computer-readable storage media of computing device 900. Processor 910 may execute any such code to provide any techniques for determining treatment of a subject based on EEG-derived biomarkers as described herein. Any other software, programs or instructions described herein may also be stored and executed by computing device 900. It will be appreciated that computer code may be applied to any aspects of methods and techniques described herein. For example, computer code may be applied to automatically determining a treatment of IS of a subject based on calculated biomarker values.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

While the techniques described herein have primarily been discussed in relation to Infantile Spasms (IS), the techniques may also be applicable in predicting the efficacy of treatment against other forms of epilepsies such as, but not limited to, Continuous spike wave during sleep, Electrical Status epilepticus in sleep, Landau Kleffner Syndrome, Status epilepticus, Lennox Gastaut Syndrome, autoimmune encephalitis related epilepsy, and/or pharmacologically refractory epilepsies; and in predicting the likelihood of relapse in a subject that has received treatment for any one or more of the above epilepsies.

Treatment of IS or another epilepsy as described herein may include application of an anti-seizure drug such as, but not limited to, steroid therapy with adrenocorticotropic hormone (ACTH) or Acthar® gel (e.g., by injection into a muscle or prednisone by mouth), Sabril® (vigabatrin), Depakote® (valproate), Topamax® (topiramate), pyridoxine (vitamin B6), Zonegran® (zonisamide), Onfi® (clobazam) or Klonopin® (clonazepam). These drugs may be used individually or in combination. For example, Sabril® (vigabatrin) may be used in combination with a steroid therapy using ACTH. Other anti-seizure drugs whose efficacy can be predicted using the methods described herein include, without limitation: adrenocorticotrophin (ACTH), acetazolamide, brivaracetam, cannabidiol, carbamazepine, clobazam, clonazepam, clorazepate, divalproex sodium, diazepam, eslicarbazepine acetate, ethosuximide, ezogabine, felbamate, gabapentin, lacosamide, lamotrigine, levetiracetam, lorazepam, methsuximide, midazolam, nitrazepam, oxcarbazepine, paraldehyde, perampanal, phenobarbital, phenobarbitone, piracetam, potassium bromide, phenytoin, pregabalin, primidone, progabide, rufinamide, stiripentol, sulthiame, tiagabine, topiramate, valproic acid, and zonizamide. The methods described herein can also be used to predict the efficacy of the combination of the drugs provided herein, e.g., without limitation, the combination of ACTH and Sabril® (vigabatrin).

Adrenocorticotropic hormone (ACTH) is a polypeptide tropic hormone produced by and secreted by the anterior pituitary gland. ACTH is an important component of the hypothalamic-pituitary-adrenal axis and is often produced in response to biological stress (along with its precursor corticotropin-releasing hormone from the hypothalamus). Its principal effects are increased production and release of cortisol by the cortex of the adrenal gland. ACTH is also related to the circadian rhythm in many organisms.

The efficacy of ACTH, particularly in high doses, for rapid and complete elimination of infantile spasms (IS) has been demonstrated in prospective controlled studies. Two forms of ACTH products are currently used in clinical trials: H.P. Acthar Gel and cosyntropin/tetracosactide/cortrosyn. Other forms of ACTH related peptide can also be used for preclinical research. Such ACTH-related peptide are listed in Table 10 below and can be used in accordance with the methods described herein.

TABLE 10
ACTH related peptides

| Name | Amino Acid Sequence |
|---|---|
| ACTH (1-10) | SYSMEHFRWG |
| ACTH (11-24) | KPVGKKRRPVKVYP |
| ACTH (1-13), human | SYSMEHFRWGKPV |
| ACTH (1-14) | SYSMEHFRWGKPVG |
| ACTH (1-17) | SYSMEHFRWGKPVGKKR |
| ACTH (1-24), human | SYSMEHFRWGKPVGKKRRPVKVYP |
| ACTH (1-24), human | SYSMEHFRWGKPVGKKRRPVKVYP |
| (10005-05) | |
| ACTH (1-39), human | SYSMEHFRWGKPVGKKRRPVKVYP |
| | NGAEDESAEAFPLEF |
| ACTH (1-39), human- | SYSMEHFRWGKPVGKKRRPVKVYP |
| 10004-005 | NGAEDESAEAFPLEF |
| ACTH (1-39), rat | SYSMEHFRWGKPVGKKRRPVKVYP |
| Adrenocorticotropic | NVAENESAEAFPLEF |
| hormone | |
| ACTH (1-4) | SYSM |
| ACTH (18-39), human | RPVKVYPNGAEDESAEAFPLEF |
| (CLIP) | |
| ACTH (22-39) | VYPNGAEDESAEAFPLEF |
| ACTH (4-10) | MEHFRWG |
| ACTH (4-11) | MEHFRWGK |
| ACTH (4-9) | MEHFRW |
| ACTH (5-10) | EHFRWG |
| ACTH (7-38), human | FRWGKPVGKKRRPVKVYPNGAEDE |
| | SAEAFPLE |
| Adrenocorticotropic | SYSMEHFRWGKPVGKK |
| Hormone (ACTH) | |
| (1-16), human | |
| Biotin-CTH (1-39), | Biotin-SYSMEHFRWGKPVGKKR |
| human | RPVKVYPNGAEDESAEAFPLEF |
| Biotin-CTH (1-39), | Biotin-SYSMEHFRWGKPVGKKR |
| human-10013-005 | RPVKVYPNGAEDESAEAFPLEF |
| [Glu10]-ACTH (1-17) | SYSMEHFRWEKPVGKKR |
| [Phe2, Nle4]-ACTH | SFS-Nle-EHFRWGKPVGKKRRPV |
| (1-24) | KVYP |
| Sauvagine | Pyr-GPPISIDLSLELLRKMIEIE |
| | KQEKEKQQAANNRLLLDTI- |
| | Amide |
| Sauvagine | Pyr-GPPISIDLSLELLRKMIEIE |
| | KQEKEKQQAANNRLLLDTI- |
| | Amide |

It has been suggested that ACTH may reduce neuronal excitability and alleviate IS by two mechanisms of action: (1) by inducing steroid release and (2) by a direct, steroid-independent action on melanocortin receptors. These combined effects may explain the robust established clinical effects of ACTH in the therapy of IS (Brunson et al., 2011). It was also observed that, not all IS patients respond to ACTH treatment. It remains unclear what factors dictate the effectiveness of ACTH in a certain IS patient.

Sabril® (vigabatrin) can be especially effective for the short-term treatment of children with infantile spasms caused by tuberous sclerosis complex (TSC). Tuberous sclerosis is a disorder that can affect the brain, skin, heart, and other parts of the body. Sabril has been associated with damage to the retina of the eye and should be used with caution in children. The retinal damage can result in permanent loss of peripheral vision, but this side effect is of more concern when the drug is used for many months. Monitoring vision in a baby on this drug is important.

An illustrative and non-limiting use of the techniques described herein will now be described. It will be appreciated that none of the experimental techniques described below are necessarily required or otherwise limiting, and are described merely to provide a descriptive example of one possible analysis procedure that may be performed utilizing the techniques described above.

### Illustrative Biomarker Study of ACTH Treatment for IS

### Demographic and Clinical Characteristics

One-hundred-fifty (83 males; 55%) out of 170 patients were included for the clinical outcome assessment study. Mean age of spasms onset was 6.6 (±3.4) months. The mean time to treatment and follow-up duration were 39 (±50) and 38 (±49) days, respectively. Fifty-one out of 150 (34%) of patients had abnormal MRI findings.

The etiology of IS was: genetic/inborn metabolic in 28 (19%), unknown in 71 (47%), and structural brain abnormality (acquired and congenital) in 51 (34%). Fifty (33%) patients had a history of epilepsy, and 57 (38%) had been using Anti-Seizure Medications (ASM) prior to ACTH treatment. During the ACTH treatment, 86 (57%) patients were receiving concomitant ASMs (Table 1).

TABLE 1
Descriptive Statistics (n: 150)

| Features | | Mean (SD) | |
|---|---|---|---|
| Age month (mean) | | 6.6 | (±3.4)months |
| Time to treatment (day) | | 39 | (±50) |
| Follow-up duration (day) | | 38 | (±49) |
| Baseline spasms frequency | | 294 | (±340)/week |
| Follow up spasms frequency | | 130 | (±341)/week |
| Gender | Male | 83 | (55.3) |
| | Female | 67 | (44.7) |
| Previous seizure history | Yes | 50 | (33.3) |
| Seizure type before the | Focal seizures | 8 | (24) |
| spasms | | | |
| | Generalized | 42 | (76) |
| | seizures | | |
| Previous AED history | Present | 57 | (38) |
| Concomitant AED | Present | 86 | (57.3) |
| Etiology | Genetic/inborn | 28 | (18.7) |
| | metabolic error | | |
| | Unknown | 71 | (47.3) |
| | Structural | 51 | (34) |
| MR results | Non- causative | 99 | (66) |
| | Causative | 51 | (34) |
| Baseline EEG features | Hypsarrhythmia | 54 | (36) |
| | Modified | 75 | (50) |
| | Hypsarrhythmia | | |
| | Without | 21 | (14) |
| | Hypsarrhythmia | | |
| Follow-up EEG features | Hypsarrhythmia | 6 | (4) |
| | Modified | 6 | (4) |
| | Hypsarrhythmia | | |
| | Without | 94 | (62.7) |
| | Hypsarrhythmia | | |
| | Normal EEG | 44 | (29.3) |
| Clinical response | Yes | 101 | (67.3) |
| | No | 49 | (32.7) |
| Relapse (if good response | Yes | 30 | (29.7) |
| exist) | | | |
| | No | 71 | (70.3) |

### Clinical Outcome

After ACTH treatment, the spasm frequency significantly decreased (median weekly: 294 vs. 130, p<0.001, Tables 1-4). One hundred-one of 150 patients (67%) were deemed responders to the ACTH treatment at follow-up assessment, with IS cessation and resolution of hypsarrhythmia (Table 2). Thirty out of 101 initial responders (30%) experienced a relapse. Clinical parameters such as age of onset, sex, MRI finding, previous seizure history, previous and concomitant AED treatment, etiology, and baseline EEG were not prognostic factors for response and relapse (Tables 2-3).

At baseline, 54 (36%) had hypsarrhythmia pattern, 75 (50%) had modified hypsarrhythmia pattern, and 21 (14%) had other abnormalities (without hypsarrhythmia) (Table 1). At the follow-up assessment, 58 patients with good response had abnormal EEG findings (abnormalities other than hypsarrhythmia, such as multifocal spikes or sharp waves, abnormal slow wave and fast activity). In terms of the EEG pattern, there was no difference between poor- and good responders, and between the patients who relapsed and the sustained responders (Tables 2-3).

TABLE 2
Comparison with respect to the treatment response

| | | Poor | | Good | | |
|---|---|---|---|---|---|---|
| | | Response | | Response | | P |
| | Test | (n): 49 | | (n): 101 | | Value |
| Spasms frequency | Student-t | 242 | (±210) | 318 | (±386.9) | NS |
| baseline/week | | | | | | |
| Spasms frequency follow | ″ | 233 | (±339) | 80 | (±332) | <0.009 |
| up/week | | | | | | |
| Age (month) | ″ | 6.1 | (±3.6) | 6.9 | (±3.3) | NS |
| Time to treatment (day) | ″ | 44.7 | (±51) | 37.1 | (±49.9) | ″ |
| Follow-up duration(day) | ″ | 42.6 | (±50) | 37.1 | (±49.6) | ″ |

| | Chi-Square test | | | |
|---|---|---|---|---|
| Gender | Male | 28 | 55 | NS |
| | Female | 21 | 46 | |
| Previous Seizure History | Present | 21 | 29 | ″ |
| Seizure type before the | Focal seizures | 4 | 4 | ″ |
| spasm | | | | |
| | Generalized | 17 | 25 | ″ |
| | seizures | | | |
| Previous AED history | Present | 23 | 34 | ″ |
| Concomitant AED | Present | 31 | 55 | ″ |
| Etiology | Genetic/metabolic | 14 | 14 | ″ |
| | Unknown | 19 | 52 | ″ |
| | Structural | 16 | 35 | ″ |
| MR finding | Normal, no cause | 33 | 66 | ″ |
| | Abnormal, probable | 16 | 35 | ″ |
| | cause | | | |
| Baseline EEG feature | Hypsarrhythmia | 18 | 36 | ″ |
| | Modified | 20 | 55 | ″ |
| | Hypsarrhythmia | | | |
| | Without | 11 | 10 | ″ |
| | Hypsarrhythmia | | | |
| Follow-up EEG feature | Hypsarrhythmia | 4 | 0 | ″ |
| | Modified | 4 | 4 | ″ |
| | Hypsarrhythmia | | | |
| | Without | 40 | 54 | <0.001 |
| | Hypsarrhythmia | | | |
| | Normal EEG | 1 | 43 | <0.001 |

TABLE 3
Comparison with respect to relapse

| | | | | Without | | |
|---|---|---|---|---|---|---|
| | | Relapse | | Relapse | | P |
| | Test | (n): 30 | | (n): 71 | | Value |
| Spasms frequency | Student-t | 311 | (±356) | 322 | (±401) | NS |
| baseline/week | | | | | | |
| Spasms frequency | ″ | 270 | (±572) | 0 | | <0.001 |
| follow up/week | | | | | | |
| Age (month) | ″ | 5.2 | (±2.8) | 7.6 | (±3.4) | NS |
| Time to treatment (day) | ″ | 29 | (±34) | 40 | (±55) | ″ |
| Follow-up duration(day) | ″ | 28 | (±35) | 40 | (±54) | ″ |

| | Chi-Square test | | | |
|---|---|---|---|---|
| Gender | Male | 11 | 44 | ″ |
| | Female | 19 | 27 | |
| Seizure type before | Focal seizures | 1 | 3 | ″ |
| the spasm | | | | |
| | Generalized | 6 | 19 | |
| | seizures | | | |
| Previous AED history | Present | 7 | 27 | ″ |
| Concomitant AED | Present | 17 | 38 | ″ |
| Etiology | Genetic/inborn | 3 | 11 | ″ |
| | metabolic error | | | |
| | Unknown | 18 | 34 | ″ |
| | Structural | 9 | 26 | ″ |
| MR finding | Normal, no cause | 21 | 45 | ″ |
| | Abnormal, probable | 9 | 26 | |
| | cause | | | |
| Baseline EEG feature | Hypsarrhythmia | 12 | 24 | ″ |
| | Modified | 17 | 38 | ″ |
| | Hypsarrhythmia | | | |
| | Without | 1 | 9 | ″ |
| | Hypsarrhythmia | | | |
| Follow-up EEG | Hypsarrhythmia | 2 | 0 | ″ |
| feature | | | | |
| | Modified | 2 | 0 | ″ |
| | Hypsarrhythmia | | | |
| | Without | 25 | 29 | <0.001 |
| | Hypsarrhythmia | | | |
| | Normal EEG | 1 | 42 | <0.001 |

TABLE 4
Quantitative EEG results at before and after ACTH treatment

| Parameters n: 50 | | | | | |
|---|---|---|---|---|---|
| (mean, SD) | Baseline EEG | | Follow-up EEG | | p Value |
| Spike Count Manual | 234 | (±115) | 112 | (±108) | <0.001 |
| Spike Count Automatic | 232 | (±117) | 113 | (±102) | <0.001 |
| Delta Power | 25.1 | (±9.7) | 17.2 | (±7.9) | <0.001 |
| Seizure frequency | 192 | (±257) | 70 | (±116) | <0.001 |

### Advanced Electrophysiological Outcome Assessment

Fifty out of these 150 patients had original EEG data available for analysis met criteria for the comparison of baseline and follow-up electrophysiological evaluation.

Upon comparison of baseline and follow-up EEG, quantitative EEG parameters decreased significantly: 234 vs. 112, p<0.001, ASCA: 232 vs. 113, p<0.001, and DP (μV):25.1 vs. 17.2, p<0.001 (Table 4). The magnitude of changes in the EEG parameters (spike quantification and slow wave activity) were more prominent in patients responders as compared to non-responders (MSC diff: 169 vs.44 spikes; p<0.001, ASCA diff: 157 vs.55 spikes; p<0.001, DP diff: 11.3 vs.2.3, Sf diff: 158 vs. 60; p<0.05 respectively in responders vs. non-responders, Table 5). Also, 26 out of 30 patients with relapse did not have a hypsarrhythmia pattern on follow-up assessment. A less prominent decrease in spike counts and DP was observed in patients who relapsed as compared to patients with sustained response (MSC diff; 76 vs. 220 p<0.001, ASCA diff; 75 vs. 202, p<0.005) and DP diff (5.4 vs. 14.6, p<0.008) (Table 6).

Baseline values of MSC, ASCA, and DP were highly correlated. Similarly, the follow-up values showed strong correlation. Baseline values of spike counts were not correlated with the follow-up values. However, the baseline DP showed a significant correlation with the follow-up DP (Spearman's correlation coefficient: 0.30, p=0.03, Table7).

Based on multiple regression analysis stepwise modeling, the baseline delta power, follow-up delta power, and follow-up spike count were significant predictors for response to ACTH treatment (Table 8). Furthermore, follow-up spike count was a predictor for relapse after an initial good response to ACTH treatment (Table 9).

TABLE 5
Comparison of change in EEG parameters and spasms
frequency among Good Responders vs. Poor Responders

| Parameters | Good | | Poor | | Between-group |
|---|---|---|---|---|---|
| (median, 25-75) | responder | | responder | | Comparison |
| (n: 50) | (n: 31) | | (n: 19) | | P value |
| Spasms frequency | | | | | |
| Baseline | 122 | (63-175) | 105 | (70-154) | >0.05 |
| Follow-up | 0 | (0-21) | 70 | (21-140) | <0.001 |
| Change | 84 | (21-175) | 43 | (−26-91) | <0.051 |
| Within-group | <0.001 | | >0.05 | | |
| Quantitative EEG Analysis | | | | | |
| Spike Count (Automatic) | | | | | |
| Baseline | 244 | (143-325) | 236 | (7-327) | >0.05 |
| Follow-up | 53 | (12-129) | 152 | (69-245) | <0.001 |
| Change | 144 | (50-266) | 47 | ([−89]-185) | <0.029 |
| Within-group | >0.05 | | >0.05 | | |
| Spike Count (Manual) | | | | | |
| Baseline | 232 | (162-332) | 242 | (84-341) | >0.05 |
| Follow-up | 48 | (3-113) | 168 | (53-252) | <0.001 |
| Change | 153 | (51-250) | 52 | (−92-104) | <0.008 |
| Within-group | <0.001 | | >0.05 | | |
| Delta Power | | | | | |
| Baseline | 24.7 | (19.5-30.9) | 21.7 | (16.5-30.7) | >0.05 |
| Follow-up | 13.4 | (9.6-19.1) | 19.5 | (14.3-25.2) | <0.005 |
| Change | 10.2 | (2.4-18.5) | 3.1 | (−2.5-9.2) | <0.004 |

| | | |
|---|---|---|
| Within-group P | <0.001 | >0.05 |

TABLE 6
Comparison of change in EEG parameters and spasms frequency
among Patients with Relapse vs Patients without Relapse

| Parameters | Patients without | | Patients with | | Between-group |
|---|---|---|---|---|---|
| (median, 25-75) | Relapse | | Relapse | | Comparison |
| (n: 31) | (n: 20) | | (n: 11) | | P value |
| Spasms frequency | | | | | |
| Baseline | 117 | (63-177) | 140 | (32-175) | >0.05 |
| Follow-up | 0 | | 42 | (18-203) | <0.0001 |
| Change | 117 | (63-177) | 11 | (−21-157) | <0.02 |
| Within-group P | <0.0001 | | 0.28 | | |
| Quantitative EEG Analysis | | | | | |
| Spike Count (Automatic) | | | | | |
| Baseline | 257 | (165-347) | 197 | (120-248) | >0.05 |
| Follow-up | 28.5 | (4.7-99.2) | 117 | (38-165) | <0.005 |
| Change | 176 | (114-296) | 50 | (−11-146) | <0.005 |
| Within-group P | <0.0001 | | 0.041 | | |
| Spike Count (Manual) | | | | | |
| Baseline | 247 | (188-361) | 187 | (137-268) | >0.05 |
| Follow-up | 10 | (2.0-92) | 86 | (42-214) | <0.01 |
| Change | 212 | (112-323) | 51 | (21-104) | <0.001 |
| Within-group P | <0.0001 | | 0.008 | | |
| Delta Power | | | | | |
| Baseline | 26.4 | (20.8-35.9) | 23.6 | (17.4-26.5) | >0.05 |
| Follow-up | 10.5 | (9.4-16.6) | 17.1 | (13.3-22.1) | <0.005 |
| Change | 14.6 | (9.2-25) | 6 | (1.1-9.6) | <0.008 |

| | | |
|---|---|---|
| Within-group P | <0.0001 | 0.013 |

TABLE 7
Correlation between different EEG parameters at baseline and at follow-up

| | | Automatic | Manual | Delta | Automatic | Manual | Delta |
|---|---|---|---|---|---|---|---|
| Spearman's Correlation | | spike count | spike count | power | spike count | spike count | power |
| Coefficient (C.C.) | | baseline | baseline | baseline | follow-up | follow-up | follow-up |
| Automatic spike | C.C. | 1 | 0.93 | 0.571 | 0.43 | −0.04 | 0.029 |
| count baseline | Significance | . | 0.0001 | 0.0001 | 0.767 | 0.978 | 0.84 |
| Manual spike | C.C. | 0.93 | 1 | 0.622 | −0.076 | −0.045 | 0.058 |
| count baseline | Significance | 0.0001 | . | 0.0001 | 0.598 | 0.757 | 0.691 |
| Delta power | C.C. | 0.571 | 0.622 | 1 | −0.131 | −0.093 | 0.306 |
| baseline | Significance | 0.0001 | 0.0001 | . | 0.365 | 0.519 | 0.031 |
| Automatic spike | C.C. | −0.043 | −0.076 | −0.131 | 1 | 0.985 | 0.498 |
| count follow-up | Significance | 0.767 | 0.598 | 0.365 | . | 0.0001 | 0.0001 |
| Manual spike | C.C. | −0.004 | −0.045 | −0.093 | 0.985 | 1 | 0.507 |
| count follow-up | Significance | 0.978 | 0.757 | 0.519 | 0.0001 | . | 0.0001 |
| Delta power | C.C. | 0.029 | 0.058 | 0.306 | 0.498 | 0.507 | 1 |
| follow-up | Significance | 0.84 | 0.691 | 0.031 | 0.0001 | 0.0001 | . |

TABLE 8
Variables in the Equation for Treatment Response

| | | | | | | | | 95% C.I. for | |
|---|---|---|---|---|---|---|---|---|---|
| | | | | | | | | EXP(B) | |
| | | B | S.E. | Wald | df | Sig. | Exp(B) | Lower | Upper |
| Step | Automatic | −0.011 | 0.004 | 8.215 | 1 | 0.004 | 0.989 | 0.981 | 0.996 |
| 1^{a} | spike | | | | | | | | |
| | follow-up | | | | | | | | |
| | Constant | 1.838 | 0.569 | 10.445 | 1 | 0.001 | 6.284 | | |
| Step | Automatic | −0.009 | 0.004 | 5.120 | 1 | 0.024 | 0.991 | 0.983 | 0.999 |
| 2^{b} | spike | | | | | | | | |
| | follow-up | | | | | | | | |
| | Delta power | −0.110 | 0.057 | 3.655 | 1 | 0.056 | 0.896 | 0.800 | 1.003 |
| | follow-up | | | | | | | | |
| | Constant | 3.511 | 1.155 | 9.247 | 1 | 0.002 | 33.484 | | |
| Step | Automatic | −0.009 | 0.005 | 3.590 | 1 | 0.058 | 0.991 | 0.982 | 1.000 |
| 3^{c} | spike | | | | | | | | |
| | follow-up | | | | | | | | |
| | Delta power | −0.230 | 0.098 | 5.524 | 1 | 0.019 | 0.794 | 0.656 | 0.962 |
| | follow-up | | | | | | | | |
| | Delta power | 0.102 | 0.052 | 3.829 | 1 | 0.050 | 1.108 | 1.000 | 1.227 |
| | baseline | | | | | | | | |
| | Constant | 3.111 | 1.405 | 4.906 | 1 | 0.027 | 22.445 | | |

^{a}Variable(s) entered on step 1: automatic spike follow-up.
^{b}Variable(s) entered on step 2: delta power follow-up.
^{c}Variable(s) entered on step 3: delta power baseline.
B: unstandardized regression coefficient,
S.E: standard error,
df: degree of freedom,
Exp (B): Odd ratio

TABLE 9
Variables in the Equation for Relapse

| | | | | | | | | 95% C.I. for | |
|---|---|---|---|---|---|---|---|---|---|
| | | | | | | | | EXP(B) | |
| | | B | S.E. | Wald | df | Sig. | Exp(B) | Lower | Upper |
| Step | automatic | 0.014 | 0.006 | 4.912 | 1 | 0.027 | 1.014 | 1.002 | 1.027 |
| 1^{a} | spike | | | | | | | | |
| | follow-up | | | | | | | | |
| | Constant | −1.762 | 0.682 | 6.676 | 1 | 0.010 | 0.172 | | |
| Step | automatic | 0.018 | 0.008 | 5.528 | 1 | 0.019 | 1.018 | 1.003 | 1.034 |
| 2^{b} | spike | | | | | | | | |
| | follow-up | | | | | | | | |
| | Delta power | −0.096 | 0.053 | 3.338 | 1 | 0.068 | 0.909 | 0.820 | 1.007 |
| | baseline | | | | | | | | |
| | Constant | 0.228 | 1.219 | 0.035 | 1 | 0.852 | 1.256 | | |

^{a}Variable(s) entered on step 1: automatic spike follow-up.
^{b}Variable(s) entered on step 2: delta power baseline.
B: unstandardized regression coefficient,
S.E: standard error,
df: degree of freedom,
Exp (B): Odd ratio

### DISCUSSION

In this retrospective descriptive study, we demonstrated a high response rate (67%) for ACTH treatment in IS patients, and this was corroborated by additional electrophysiological parameters, such as spike frequency and delta power burden beyond the hypsarrhythmia disappearances.

### Response to Treatment

Upon follow-up assesment after day 14 of treatment, spasm cessation and hypsarrhythmia resolution were observed in 101 patients (67%), and relapse rate was 30%. The response and relapse rates were comparable to previous reports (spams cessation rates: 49-76% and EEG resolution: 22-87%), relapse rate: 15-41%.

According to previous studies, etiology was the most important predictor of outcomes, and unknown etiology had a better response rate to ACTH treatment. In the current study, unknown etiology was the most frequent etiology and patients with unknown etiology had a good responder rate, but the comparisons between different etiologies were not statistically significant. Early time to treatment, no previous history of epilepsy, and no prior AED treatment were noted to be prognostic factors for good response in selected previous studies. In the current study, we did not find any significant effects of clinical factors on the treatment outcome, possibly due to low numbers.

### EEG Evaluation

Different criteria have been utilized to determine response to treatment in previous studies. Our EEG responder criteria are consistent with the criteria used in several previous studies, comprising IS freedom for at least seven consecutive days during treatment, and disappearance of hypsarrhythmia on follow-up EEG assessment. Although the presence of hypsarrhythmia has been considered crucial for the diagnosis and evaluating treatment response in IS, hypsarrhythmia may not be present at diagnosis in 25% of cases. Lack of hypsarrhythmia may be seen in patients with prior AED treatment, the early phase of IS, and late onset of spasms. In our study, 21 out of 150 patients (14%) did not show a hypsarrhythmia pattern on EEG at the first presentation, and 26 out of 30 patients with relapse did not have hypsarrhythmia in follow up EEGs. Thus, it does not seem sufficient to only rely on hypsarrhythmia for the diagnosis of IS and determining relapse. Our findings highlight the importance of new quantitive biomarkers for evaluating EEGs in IS.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A method of adapting treatment of a subject having infantile spasms (IS), the method comprising:
obtaining electroencephalogram (EEG) data of the subject;
determining a measure of delta power of the EEG data and/or a measure of spike frequency of the EEG data; and
determining subsequent treatment of the infantile spasms of the subject based at least in part on the determined measure of delta power of the EEG data and/or measure of spike frequency of the EEG data.

2. The method of claim 1, wherein the infantile spasms of the subject have previously been treated with one or more medications, and wherein determining the subsequent treatment of the infantile spasms of the subject comprises determining a new dose of at least one of the one or more medications.

3. The method of claim 1, wherein the infantile spasms of the subject have previously been treated with a first medication, and wherein determining the subsequent treatment of the infantile spasms of the subject comprises selecting a second medication for subsequent treatment of the subject.

4. The method of claim 3, wherein determining the subsequent treatment of the infantile spasms of the subject further comprises determining to cease subsequent treatment of the subject with the first medication.

5. The method of claim 1, further comprising:
comparing:
the determined measure of delta power of the EEG data with a previously determined measure of delta power of the subject; and/or
the determined measure of spike frequency of the EEG data with a previously determined measure of spike frequency of the subject, and
wherein the subsequent treatment of the infantile spasms of the subject is determined based at least in part on a result of said comparison.

6. The method of claim 5, wherein determining the subsequent treatment of the infantile spasms of the subject comprises comparing the result of said comparison with a threshold value.

7. The method of claim 1, wherein the infantile spasms of the subject have previously been treated, and wherein determining the subsequent treatment of the infantile spasms of the subject comprises determining a likelihood of relapse of the subject.

8. The method of claim 7, wherein,
when the determined likelihood of relapse of the subject is above a first value, selecting a new treatment for the infantile spasms of the subject, and
when the determined likelihood of relapse of the subject is below a second value, maintaining a current treatment for the infantile spasms of the subject.

9. The method of claim 1, comprising determining the measure of delta power of the EEG data, and wherein determining the measure of delta power of the EEG data includes calculating a spectral power of the EEG data in a first frequency band.

10. The method of claim 9, wherein the first frequency band is a range of frequencies between an upper frequency and a lower frequency, wherein the upper frequency is no greater than 5 Hz, and wherein the lower frequency is greater than 0 Hz.

11. The method of claim 1, comprising determining the measure of delta power of the EEG data, and wherein determining the measure of delta power of the EEG data includes performing a Fast Fourier Transform (FFT) of the EEG data.

12. The method of claim 1, comprising determining the measure of spike frequency of the EEG data, and wherein determining the measure of spike frequency of the EEG data includes identifying a plurality of amplitude spikes in the EEG data, the amplitude spikes being a deviation from a baseline amplitude above an amplitude threshold.

13. The method of claim 12, wherein the amplitude threshold is at least three times the baseline amplitude.

14. The method of claim 1, comprising determining the measure of spike frequency of the EEG data, and wherein determining the measure of spike frequency of the EEG data includes calculating a plurality of wavelet coefficients of the EEG data.

15. The method of claim 1, wherein the subsequent treatment of the infantile spasms of the subject comprises ACTH therapy.

16. The method of claim 15, wherein the ACTH therapy comprises application of Acthar® gel.

17. The method of claim 1, wherein the subject is less than one year of age.

18. The method of claim 1, wherein the obtained EEG data of the subject comprises non-rapid-eye movement sleep (NREMS) EEG data.

19. The method of claim 1, further comprising treating the infantile spasms of the subject according to the determined subsequent treatment of the infantile spasms of the subject.

20. A non-transitory computer readable medium comprising instructions that, when executed by at least one processor, perform a method of adapting treatment of a subject having infantile spasms (IS), the method comprising:
accessing electroencephalogram (EEG) data of the subject;
determining, using the at least processor, a measure of delta power of the EEG data and/or a measure of spike frequency of the EEG data; and
determining, using the at least processor, subsequent treatment of the infantile spasms of the subject based at least in part on the determined measure of delta power of the EEG data and/or measure of spike frequency of the EEG data.

21. The non-transitory computer readable medium of claim 20, wherein determining the subsequent treatment of the infantile spasms of the subject further comprises determining to cease subsequent treatment of the subject with a first medication.

22. The non-transitory computer readable medium of claim 20, wherein the method further comprises:
comparing:
the determined measure of delta power of the EEG data with a previously determined measure of delta power of the subject; and/or
the determined measure of spike frequency of the EEG data with a previously determined measure of spike frequency of the subject, and
wherein the subsequent treatment of the infantile spasms of the subject is determined based at least in part on a result of said comparison.

23. The non-transitory computer readable medium of claim 22, wherein determining the subsequent treatment of the infantile spasms of the subject comprises comparing the result of said comparison with a threshold value.

24. The non-transitory computer readable medium of claim 20, wherein determining the subsequent treatment of the infantile spasms of the subject comprises determining a likelihood of relapse of the subject.

25. The non-transitory computer readable medium of claim 24, wherein,
when the determined likelihood of relapse of the subject is above a first value, selecting a new treatment for the infantile spasms of the subject, and
when the determined likelihood of relapse of the subject is below a second value, maintaining a current treatment for the infantile spasms of the subject.

26. The non-transitory computer readable medium of claim 20, wherein the method comprises determining the measure of delta power of the EEG data, and wherein determining the measure of delta power of the EEG data includes calculating a spectral power of the EEG data in a first frequency band.

27. The non-transitory computer readable medium of claim 26, wherein the first frequency band is a range of frequencies between an upper frequency and a lower frequency, wherein the upper frequency is no greater than 5 Hz, and wherein the lower frequency is greater than 0 Hz.

28. The non-transitory computer readable medium of claim 20, wherein the method comprises determining the measure of delta power of the EEG data, and wherein determining the measure of delta power of the EEG data includes performing a Fast Fourier Transform (FFT) of the EEG data.

29. The non-transitory computer readable medium of claim 20, wherein the method comprises determining the measure of spike frequency of the EEG data, and wherein determining the measure of spike frequency of the EEG data includes identifying a plurality of amplitude spikes in the EEG data, the amplitude spikes being a deviation from a baseline amplitude above an amplitude threshold.

30. The non-transitory computer readable medium of claim 29, wherein the amplitude threshold is at least three times the baseline amplitude.

31. The non-transitory computer readable medium of claim 20, wherein the method comprises determining the measure of spike frequency of the EEG data, and wherein determining the measure of spike frequency of the EEG data includes calculating a plurality of wavelet coefficients of the EEG data.

32. The non-transitory computer readable medium of claim 20, wherein the subsequent treatment of the infantile spasms of the subject comprises ACTH therapy.
